(19) United States
(12) Patent Application Publication
(10) Pub. No.: US 2026/0272267 A1
(43) Pub. Date: Sep. 17, 2026

(54) POLYMERIC MATERIAL FUSION JOINING METHOD, TUBE JOINT STRUCTURE, AND ENDOSCOPE

(71) Applicant: HUNAN VATHIN MEDICAL INSTRUMENT CO., LTD., Xiangtan (CN)

(72) Inventors: Wenjun MO, Xiangtan (CN); Peng TANG, Xiangtan (CN)

(73) Assignee: HUNAN VATHIN MEDICAL INSTRUMENT CO., LTD., Xiangtan (CN)

(21) Appl. No.: 19/668,958

(22) Filed: May 6, 2026

Related U.S. Application Data
(63) Continuation of application No. PCT/CN2024/130198, filed on Nov. 6, 2024.

(30) Foreign Application Priority Data
Nov. 6, 2023 (CN) 202311463838.X

(51) Int. Cl. A61B 1/00; B29C 65/68; B29C 65/78; B29L 23/00

(52) U.S. Cl. CPC A61B 1/0011; B29C 65/68; B29C 65/7838; B29L 2023/007

(57) ABSTRACT

A polymeric material fusion joining method, a tube joint structure, and an endoscope are provided. The method includes a first step of abutting a base tube and a mating member to be joined to each other to form a joint seam; a second step of fitting a tubular interlocking sleeve spanning across the joint seam over an outer periphery of the base tube and the mating member; a third step of heating the interlocking sleeve to cause thermal contraction thereof, such that at least a portion of the material of the interlocking sleeve infiltrates into an entire circumferential wall of the base tube and the mating member and fills the joint seam over an entire circumference thereof; and a fourth step of allowing the interlocking sleeve to cool and solidify to form an interlocking engagement with the base tube and the mating member, respectively, and to seal the joint seam.

### CROSS-REFERENCE TO RELATED APPLICATION

This application is a U.S. National Stage application of International Application No. PCT/CN 2024/130198, filed on Nov. 6, 2024, which claims the benefit of priority to Chinese Patent Application No. CN202311463838X, filed on Nov. 6, 2023. The entire disclosure of the aforementioned application is incorporated herein by reference.

### TECHNICAL FIELD

The present disclosure relates to the field of endoscope technology, and more particularly, to a polymeric material fusion joining method, a tube joint structure, and an endoscope.

### BACKGROUND

When using an endoscope to treat or examine a diseased site within the human body, it is desired to maintain the hygiene of the endoscope. Therefore, after each use or after a certain storage period has elapsed, the endoscope needs to undergo rigorous disinfection and sterilization.

In current endoscopes, after prolonged use and repeated disinfection and sterilization cycles, the butt-joined ends of two tube members secured by adhesive dispensing are prone to seal failure, resulting in water ingress into the device and consequently leading to problems such as loss of illumination and loss of imaging.

### SUMMARY

An object of the present disclosure is to provide a polymeric material fusion joining method, a tube joint structure, and an endoscope, so as to solve the problem of seal failure that is prone to occur at the butt-joined ends of tube members.

The present disclosure is realized through the following technical solutions:

The present disclosure provides a polymeric material fusion joining method, including:

a first step of abutting a base tube and a mating member that are to be joined to each other so as to form a joint seam at an abutment junction thereof;
a second step of fitting a tubular interlocking sleeve, which spans across the joint seam, over an outer periphery of the base tube and the mating member;
a third step of heating the interlocking sleeve to cause thermal contraction thereof, such that at least a portion of the material of the interlocking sleeve infiltrates into the entire circumferential wall of the base tube and the mating member and fills the joint seam over an entire circumference thereof; and
a fourth step of allowing the interlocking sleeve to cool and solidify so as to form an interlocking engagement with the base tube and the mating member, respectively, and to seal the joint seam.

According to the above method, the interlocking sleeve made of a polymeric material has the ability to contract upon heating. By subjecting the interlocking sleeve wrapping the joint seam to thermal contraction, the material of the interlocking sleeve in a molten state is caused, under the contraction force, to enter the structures of the base tube and the mating member to form an interlocking engagement and fuse into an integral whole, thereby achieving a seamless butt joint between the base tube and the mating member. The use of the interlocking sleeve to form an interlocking structure between the base tube and the mating member avoids the use of adhesive dispensing for connection, which makes the connection structure at the abutment junction of the base tube and the mating member reliable and prevents seal failure caused by detachment of the seal at the abutment junction regardless of the duration of use and repeated disinfection and sterilization. Meanwhile, since the interlocking sleeve is at least partially embedded within the base tube and the mating member, the outer diameter at the abutment junction of the base tube and the mating member can be controlled while ensuring reliability of the connection between the base tube and the mating member, thereby avoiding exceeding dimensional tolerances.

In a further embodiment, the mating member is a tube, a tube fitting, or an end cap.

In a further embodiment, the interlocking sleeve is a structural component made of Pebax material.

In a further embodiment, the base tube is a structural component made of any one of Pebax, PTFE, LDPE, and TPU.

In a further embodiment, the mating member is a structural component made of any one of Pebax, PTFE, LDPE, and TPU.

In a further embodiment, in the third step, the interlocking sleeve is heated by heating the ambient environment surrounding the interlocking sleeve.

In a further embodiment, in the third step, all of the material of the interlocking sleeve infiltrates into the entire circumferential wall of the base tube and the mating member and fills the joint seam over the entire circumference thereof, such that an outer peripheral surface of the interlocking sleeve is flush with outer peripheral surfaces of the base tube and the mating member.

According to the above method, causing all of the material of the interlocking sleeve to infiltrate into the entire circumferential wall of the base tube and the mating member and to fill the joint seam over the entire circumference, such that the outer peripheral surface of the interlocking sleeve is flush with the outer peripheral surfaces of the base tube and the mating member, avoids the formation of a stepped protrusion at the abutment junction of the base tube and the mating member and does not increase the outer diameter dimension at this location.

In a further embodiment, in the second step, a tubular heat-shrink film spanning across the joint seam is fitted over the outer periphery of the interlocking sleeve, wherein a melting point of the heat-shrink film is higher than a melting point of the interlocking sleeve; in the third step, the heat-shrink film is heated to cause thermal contraction thereof, so as to apply a contraction force to the interlocking sleeve through the contraction; in the fourth step, the heat-shrink film is allowed to cool and solidify; and the polymeric material fusion joining method further includes a fifth step of peeling off the heat-shrink film.

According to the above method, after fitting the heat-shrink film over the outer periphery of the interlocking sleeve, the contraction of the heat-shrink film applies a contraction force to the interlocking sleeve, which assists the interlocking sleeve in the molten state to infiltrate more rapidly into the interior of the base tube and the mating member. This makes it possible to increase the amount of infiltration of the interlocking sleeve within a given period of time to achieve better connection quality between the base tube and the mating member, or to form a connection of a given quality between the base tube and the mating member in a shorter period of time, so as to avoid prolonged heating that would affect the smoothness of the inner peripheral surfaces of the base tube and the mating member.

In a further embodiment, in the second step, a heat-shrink film having a length greater than a length of the interlocking sleeve is selected, such that the heat-shrink film completely covers the interlocking sleeve in an axial direction.

In a further embodiment, the heat-shrink film is a structural component made of FEP material.

In a further embodiment, a shrink ratio of the heat-shrink film is greater than 1.6:1.

In a further embodiment, in the third step, heating of the interlocking sleeve and the heat-shrink film is stopped when an inner peripheral surface of the heat-shrink film at a portion thereof covering the interlocking sleeve comes into contact with the outer peripheral surfaces of the base tube and the mating member.

According to the above method, stopping the heating of the interlocking sleeve and the heat-shrink film when the inner peripheral surface of the heat-shrink film at the portion covering the interlocking sleeve comes into contact with the outer peripheral surfaces of the base tube and the mating member ensures that the interlocking sleeve is precisely and fully embedded within the base tube and the mating member, and avoids continued heating that would cause the interlocking sleeve to infiltrate excessively into the base tube and the mating member, which would result in reduced connection strength.

In a further embodiment, in the second step, a mandrel spanning at least across the interlocking sleeve is inserted through the base tube and the mating member to support inner peripheral surfaces of the base tube and the mating member.

The present disclosure further provides a tube joint structure produced by the above method. The tube joint structure includes an interlocking sleeve, and a base tube and a mating member abutted against each other, wherein the base tube and the mating member form a joint seam at an abutment junction thereof. At least a portion of the material of the interlocking sleeve is embedded from an outer side into the entire circumferential wall of the base tube and the mating member to form an interlocking structure, and fills the joint seam from the outer side to seal the joint seam.

The present disclosure further provides an endoscope including an insertion section, wherein the insertion section has a plurality of the above tube joint structures.

The present disclosure has the following advantages and beneficial effects:

In the present disclosure, the interlocking sleeve made of a polymeric material has the ability to contract upon heating. By subjecting the interlocking sleeve wrapping the joint seam to thermal contraction, the material of the interlocking sleeve in a molten state is caused, under the contraction force, to enter the structures of the base tube and the mating member to form an interlocking engagement and fuse into an integral whole, thereby achieving a seamless butt joint between the base tube and the mating member. The use of the interlocking sleeve to form an interlocking structure between the base tube and the mating member avoids the use of adhesive dispensing for connection, which makes the connection structure at the abutment junction of the base tube and the mating member reliable and prevents seal failure caused by detachment of the seal at the abutment junction regardless of the duration of use and repeated disinfection and sterilization. Meanwhile, since the interlocking sleeve is at least partially embedded within the base tube and the mating member, the outer diameter at the abutment junction of the base tube and the mating member can be controlled while ensuring reliability of the connection between the base tube and the mating member, thereby avoiding exceeding dimensional tolerances.

### BRIEF DESCRIPTION OF THE DRAWINGS

To more clearly illustrate the technical solutions of the embodiments of the present disclosure or the prior art, the following briefly introduces the drawings used in the description of the embodiments or the prior art. It is apparent that the drawings described below are merely some embodiments of the present disclosure, and that other drawings may be derived from these drawings by those of ordinary skill in the art without creative effort.

FIG. 1 illustrates the base tube and the mating member in an abutted configuration.

FIG. 2 illustrates a structure in which the interlocking sleeve is fitted over the outer periphery at the joint seam, based on FIG. 1.

FIG. 3 illustrates the thermal contraction direction of the interlocking sleeve when the interlocking sleeve of the structure shown in FIG. 2 is heated.

FIG. 4 illustrates the tube joint structure with the interlocking sleeve fully embedded within the base tube and the mating member.

FIG. 5 illustrates a structure in which the heat-shrink film is fitted over the outer periphery of the interlocking sleeve, based on FIG. 2.

FIG. 6 illustrates the thermal contraction directions of the heat-shrink film and the interlocking sleeve when the heat-shrink film and the interlocking sleeve of the structure shown in FIG. 5 are heated, with a mandrel inserted through the base tube and the mating member.

FIG. 7 illustrates the tube joint structure with the interlocking sleeve fully embedded within the base tube and the mating member, prior to peeling off the heat-shrink film.

### REFERENCE NUMERALS

1, base tube; 2, mating member; 3, joint seam; 4, interlocking sleeve; 5, heat-shrink film; 6, mandrel.

### DETAILED DESCRIPTION

To make the objects, technical solutions, and advantages of the present disclosure clearer, the technical solutions of the present disclosure will be described in detail below. It is apparent that the described embodiments are merely some, but not all, embodiments of the present disclosure. All other embodiments obtained by those of ordinary skill in the art based on the embodiments of the present disclosure without creative effort fall within the scope of protection of the present disclosure.

In the description of the present disclosure, it should be noted that, unless otherwise specified, “a plurality of” means two or more. The terms “upper,” “lower,” “left,” “right,” “inner,” “outer,” “front end,” “rear end,” “head,” “tail,” and the like, indicating orientations or positional relationships, are based on the orientations or positional relationships shown in the drawings, and are merely for convenience of description and simplification of the description, rather than indicating or implying that the referenced device or element must have a particular orientation or be constructed and operated in a particular orientation. Therefore, these terms should not be construed as limiting the present disclosure. Additionally, the terms “first,” “second,” “third,” and the like are used for descriptive purposes only and are not to be construed as indicating or implying relative importance.

In the description of the present disclosure, it should also be noted that, unless otherwise expressly specified and defined, the terms “mounted,” “connected,” and “coupled” should be understood broadly and may include, for example, a fixed connection, a detachable connection, or an integral connection; a mechanical connection or an electrical connection; a direct connection or an indirect connection through an intermediary. Those of ordinary skill in the art may understand the specific meaning of the above terms in the context of the present disclosure depending on the specific circumstances.

To solve the problem of seal failure that is prone to occur at the butt-joined ends of tube members in an endoscope, the inventor conducted research on the sealing structure at the butt-joined ends of the tube members. During the research, the inventor found that the cause of seal failure at the butt-joined ends of the relevant tube members of the endoscope is the failure and detachment of the adhesive used to secure and seal the connection ends.

This is because, at the current stage, the fixed sealing of the butt-joined ends of tube members in endoscopes is mostly achieved by connecting the tube members to mating members by means of adhesive dispensing, so as to provide a watertight seal between the tube members and the mating members. In some cases, a combined approach of adhesive dispensing and heat-shrink tubing is used, in which a heat-shrink tube is fitted over the outer periphery of the tube member and the mating member, the heat-shrink tube is thermally contracted to wrap around and compress the tube member and the mating member together, and adhesive is additionally dispensed at the contact locations between the heat-shrink tube and the tube member and the mating member, so as to provide a watertight seal between the tube member and the mating member. After prolonged use of the endoscope, the adhesive ages due to long-term use and repeated disinfection and sterilization, leading to detachment and resulting in seal failure between the tube member and the mating member. This causes water ingress into the device and consequently leads to problems such as loss of illumination and loss of imaging. Furthermore, the combined approach of adhesive dispensing and heat-shrink tubing tends to cause the outer diameter of the product to exceed dimensional tolerances due to the wrapping of the tube member by the heat-shrink tube. Therefore, the combined approach of adhesive dispensing and heat-shrink tubing is generally less frequently adopted.

In one aspect, the present application provides a polymeric material fusion joining method capable of solving the problem of seal failure that is prone to occur at the butt-joined ends of endoscope tube members, as illustrated in FIGS. 1 through 7:

The polymeric material fusion joining method of the present embodiment abandons the conventional approaches of adhesive dispensing and combined heat-shrink tube coverage with adhesive dispensing for joining the base tube 1 and the mating member 2, and specifically employs a fusion approach to join the base tube 1 and the mating member 2, so as to avoid the problem of seal failure caused by adhesive aging.

Specifically, the polymeric material fusion joining method employs only an interlocking sleeve 4 made of a polymeric material as a connecting member. Upon heating, the molecular structure of the interlocking sleeve 4 made of such material can be disrupted and, upon cooling, reorganized, providing excellent plasticity. By heating the interlocking sleeve to a molten state and utilizing its ability to thermally contract, the material is caused to infiltrate into the interior of the base tube 1 and the mating member 2 to form an interlocking engagement, such that the base tube 1 and the mating member 2 are firmly secured together through the infiltrated portions of the interlocking sleeve 4, and the abutment junction of the base tube 1 and the mating member 2 is sealed, thereby solving the problem of seal failure.

In the present embodiment, the mating member 2 is configured to be joined to the base tube 1 to form a tubular structure having a particular functionality. The mating member 2 may be a tube of the same specification for extending the base tube 1, may be a tube fitting for joining with the base tube 1 so as to achieve a particular function of a device utilizing the channel of the base tube 1, or may be an end cap for occluding an end of the base tube 1.

The following description will continue with the mating member 2 being a tube as an example.

The polymeric material fusion joining method generally includes the following steps:

First Step: The base tube 1 and the mating member 2 to be joined are prepared and brought into abutment with each other, with the base tube 1 and the mating member 2 forming a circumferential joint seam 3 at the abutment junction. In this step, the base tube 1 and the mating member 2 may be abutted together as illustrated in FIG. 1, such that the abutment junction of the base tube 1 and the mating member 2 is a linearly extending straight segment. Alternatively, the base tube 1 and the mating member 2 may be abutted together at an included angle, such that the abutment junction of the base tube 1 and the mating member 2 is a curved segment having a given bending angle.

It should be noted that the following description will continue with the manner in which the base tube 1 and the mating member 2 are abutted together as illustrated in FIG. 1 as an example.

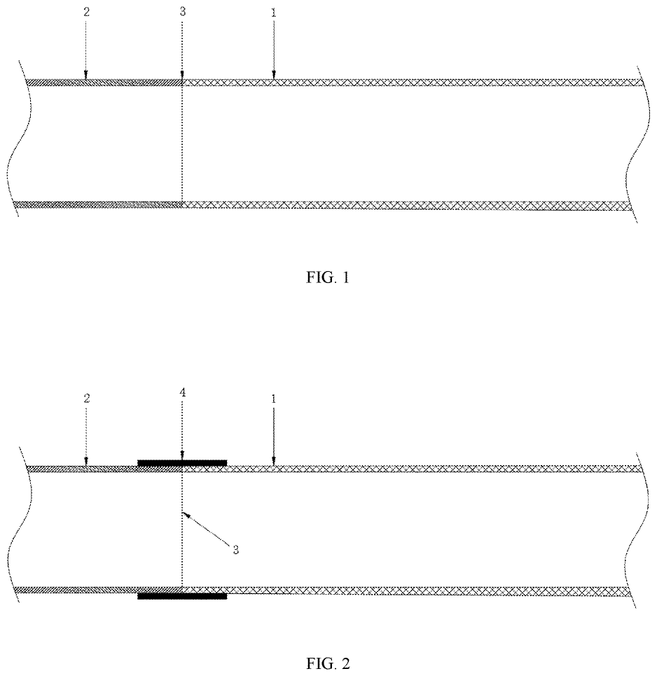

Second Step: A tubular interlocking sleeve 4 is fitted over the outer periphery of the base tube 1 and the mating member 2 such that the interlocking sleeve 4 spans across the joint seam 3, forming the structure illustrated in FIG. 2. In this step, the interlocking sleeve 4 may be pre-fitted over the base tube 1 or the mating member 2, and after the base tube 1 and the mating member 2 have been brought into abutment, the interlocking sleeve 4 is then moved to the abutment junction of the base tube 1 and the mating member 2.

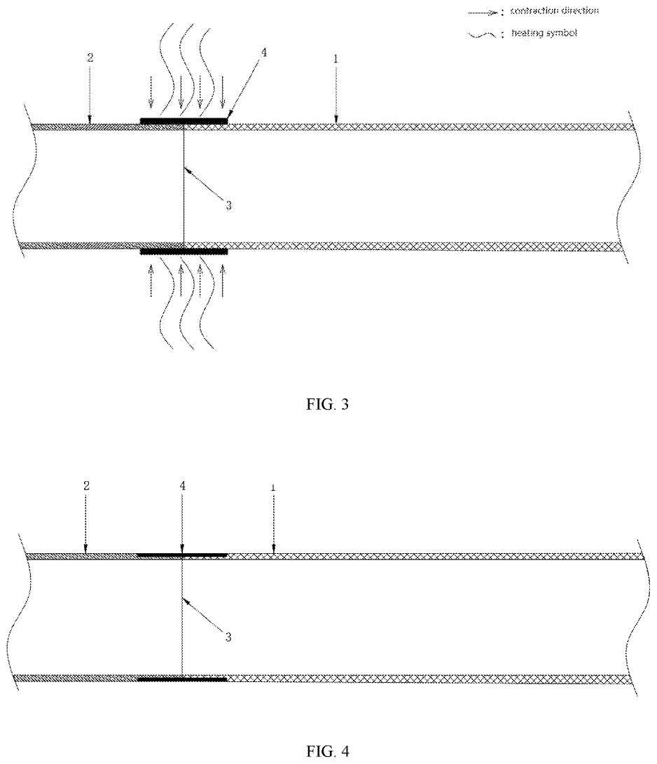

Third Step: As illustrated in FIG. 3, a heat source is disposed externally of the interlocking sleeve 4 to heat the interlocking sleeve 4 from the exterior, causing the interlocking sleeve 4 to reach a molten state in which thermal contraction occurs. In this step, at least a portion of the material of the interlocking sleeve 4 spanning across the joint seam 3 infiltrates circumferentially from the outer side toward the inner side into the entire circumferential wall of the base tube 1 and the mating member 2 along the contraction direction illustrated in FIG. 3, and simultaneously fills the joint seam 3 over the entire circumference. In this step, the molecular structure of the material of the interlocking sleeve 4 on the inner side thereof, which is located on the outer side of the base tube 1 and the mating member 2, is disrupted in the molten state and, under the contraction pressure, infiltrates into the surface micropores and internal interstices of the base tube 1 and the mating member 2 to form an interlocking engagement.

Fourth Step: Heating of the interlocking sleeve 4 is stopped, and the interlocking sleeve 4 is allowed to gradually cool and solidify. The molecular structure of the cooled interlocking sleeve 4 is reorganized, and the portions of material that have infiltrated into the base tube 1 and the mating member 2 respectively form an interlocking engagement with the base tube 1 and the mating member 2, such that the base tube 1 and the mating member 2 are fixedly joined through the interlocking sleeve 4. The material that has infiltrated into the joint seam 3 solidifies to seal the joint seam 3.

After a stable connection structure is formed among the interlocking sleeve 4, the base tube 1, and the mating member 2, the portions of the interlocking sleeve 4 embedded within the base tube 1 and the mating member 2 do not affect the smoothness of the inner peripheral surfaces of the base tube 1 and the mating member 2, thereby ensuring that the base tube 1 and the mating member 2 can provide their normal functions. Generally, the wall thickness of the interlocking sleeve 4 is less than the wall thickness of the base tube 1 and the mating member 2, such that the interlocking sleeve 4 does not affect the smoothness of the inner peripheral surfaces of the base tube 1 and the mating member 2 even when fully embedded within the base tube 1 and the mating member 2, unless the base tube 1 and the mating member 2 are also heated to a fully molten state.

In this polymeric material fusion joining method, the interlocking sleeve 4 made of a polymeric material has the ability to contract upon heating. By subjecting the interlocking sleeve 4 wrapping the joint seam 3 to thermal contraction, the material of the interlocking sleeve 4 in a molten state is caused, under the contraction force, to enter the structures of the base tube 1 and the mating member 2 to form an interlocking engagement and fuse into an integral whole, thereby achieving a seamless butt joint between the base tube 1 and the mating member 2. The use of the interlocking sleeve 4 to form an interlocking structure between the base tube 1 and the mating member 2 avoids the use of adhesive dispensing for connection, which makes the connection structure at the abutment junction of the base tube 1 and the mating member 2 reliable and prevents seal failure caused by detachment of the seal at the abutment junction regardless of the duration of use and repeated disinfection and sterilization. Meanwhile, since the interlocking sleeve 4 is at least partially embedded within the base tube 1 and the mating member 2, the outer diameter at the abutment junction of the base tube 1 and the mating member 2 can be effectively controlled while ensuring the reliability of the connection between the base tube 1 and the mating member 2, thereby effectively avoiding exceeding dimensional tolerances and facilitating application to existing devices such as endoscopes.

According to some optional embodiments, the interlocking sleeve 4 is a tubular structural component made of Pebax (polyether block amide resin) material. However, the material of the interlocking sleeve 4 is not limited thereto, and may also be other similar existing polymeric materials that have the ability to be thermally softened upon heating and whose molecular structure can be disrupted upon heating and reorganized upon cooling.

The base tube 1 may be a structural component made of any one of Pebax, PTFE (polytetrafluoroethylene), LDPE (low-density polyethylene), and TPU (thermoplastic polyurethane), but may also be made of other polymeric materials having similar properties.

The mating member 2 may be a structural component made of any one of Pebax, PTFE (polytetrafluoroethylene), LDPE (low-density polyethylene), and TPU (thermoplastic polyurethane), but may also be made of other polymeric materials having similar properties.

The base tube 1 and the mating member 2 may be made of the same material or of different materials.

By way of example, the base tube 1, the mating member 2, and the interlocking sleeve 4 are all structural components made of Pebax material. In this example, the base tube 1 and the mating member 2 are made of the same Pebax material for joining, and the interlocking sleeve 4 for fusion is also made of Pebax, which allows the interlocking sleeve 4 to fuse well with the base tube 1 and the mating member 2 at a given temperature, thereby effectively avoiding the formation of relatively sharp stepped protrusions that would affect the smoothness of the outer peripheral surface of the product.

By way of example, the base tube 1 is made of PTFE, the mating member 2 is made of Pebax, and the interlocking sleeve 4 for fusion is made of Pebax.

By way of example, the base tube 1 is made of LDPE, the mating member 2 is made of TPU, and the interlocking sleeve 4 for fusion is made of Pebax.

According to some optional embodiments, in the third step, the interlocking sleeve 4 is heated by a heating device. All of the material of the interlocking sleeve 4 in the molten state infiltrates from the outer side inward into the entire circumferential wall of the base tube 1 and the mating member 2 and fills the joint seam 3 over the entire circumference, forming the structure illustrated in FIG. 4.

Preferably, after the interlocking sleeve 4 has been fully embedded within the base tube 1 and the mating member 2, the outer peripheral surface of the interlocking sleeve 4 is flush with the outer peripheral surfaces of the base tube 1 and the mating member 2, such that the interlocking sleeve 4 is exactly fully embedded within the base tube 1 and the mating member 2.

In the present embodiment, causing all of the material of the interlocking sleeve 4 to infiltrate into the entire circumferential wall of the base tube 1 and the mating member 2 and to fill the joint seam 3 over the entire circumference, such that the outer peripheral surface of the interlocking sleeve 4 is flush with the outer peripheral surfaces of the base tube 1 and the mating member 2, avoids the formation of a radial protrusion, such as a stepped protrusion, at the abutment junction of the base tube 1 and the mating member 2, and does not increase the outer diameter dimension at this location.

According to some optional embodiments, in the second step, after the interlocking sleeve 4 has been positioned, a tubular heat-shrink film 5 is further fitted over the outer periphery of the interlocking sleeve 4 such that the heat-shrink film 5 spans across the joint seam 3, forming the structure illustrated in FIG. 5. In this step, the heat-shrink film 5 may be pre-fitted over the base tube 1 or the mating member 2, and after the base tube 1 and the mating member 2 have been brought into abutment and the position of the interlocking sleeve 4 has been adjusted, the heat-shrink film 5 is then moved to the outer periphery of the interlocking sleeve 4. The melting point of the material of the heat-shrink film 5 needs to be higher than the melting point of the material of the interlocking sleeve 4.

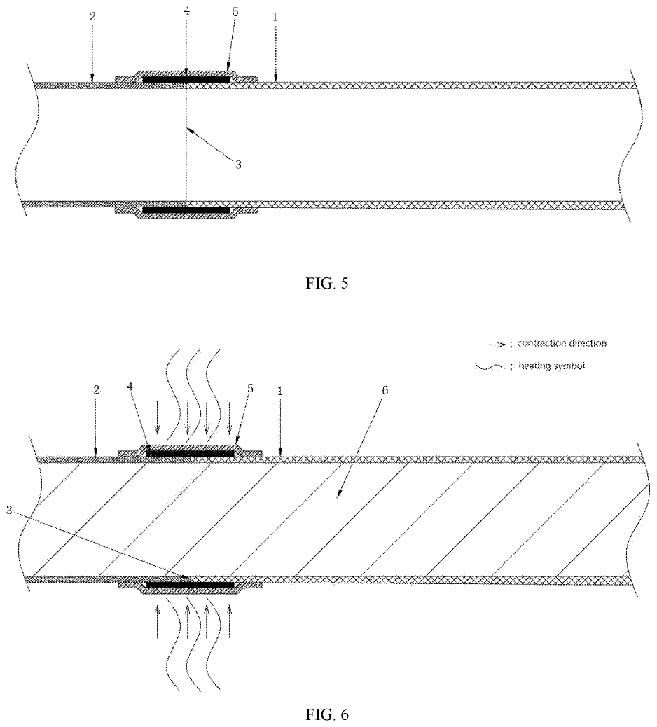

In the third step, as illustrated in FIG. 6, a heat source is disposed externally of the heat-shrink film 5 to simultaneously heat the heat-shrink film 5 and the interlocking sleeve 4 from the exterior, causing both to reach a molten state in which thermal contraction occurs along the contraction direction illustrated in FIG. 6, so as to apply an additional inward contraction force to the interlocking sleeve 4 through the contraction of the heat-shrink film 5.

In the fourth step, the heat-shrink film 5 and the interlocking sleeve 4 are allowed to cool and solidify together, and the material of the interlocking sleeve 4 that has infiltrated into the base tube 1 and the mating member 2 forms an interlocking structure with the base tube 1 and the mating member 2.

After the fourth step, a fifth step is performed to peel off the cooled and solidified heat-shrink film 5.

In the third step, the heat source is used to heat the interlocking sleeve 4 and the heat-shrink film 5, causing the interlocking sleeve 4 and the heat-shrink film 5 to reach a molten state in which thermal contraction occurs. The heat-shrink film 5 spanning across the joint seam 3 applies an inward contraction pressure to the interlocking sleeve 4 in this region, assisting the material of the interlocking sleeve 4 to infiltrate into the surface micropores and internal interstices of the base tube 1 and the mating member 2.

Preferably, in the second step, the length of the heat-shrink film 5 is greater than the length of the interlocking sleeve 4, and the heat-shrink film 5 completely covers the interlocking sleeve 4 in the axial direction. In other words, at least one axial end of the heat-shrink film 5 extends beyond the corresponding end of the interlocking sleeve 4, and the other axial end is at least flush with the corresponding end of the interlocking sleeve 4.

In the present embodiment, after the heat-shrink film 5 is fitted over the outer periphery of the interlocking sleeve 4, the contraction of the heat-shrink film 5 applies a contraction force to the interlocking sleeve 4, which assists the interlocking sleeve 4 in the molten state to infiltrate more rapidly into the interior of the base tube 1 and the mating member 2. This makes it possible to increase the amount of infiltration of the interlocking sleeve 4 within a given period of time to achieve better connection quality between the base tube 1 and the mating member 2, or to form a connection of a given quality between the base tube 1 and the mating member 2 in a shorter period of time, so as to avoid excessive deformation of the base tube 1 and the mating member 2 due to prolonged heating or infiltration of the material of the interlocking sleeve 4 into deeper positions within the base tube 1 and the mating member 2 that would affect the smoothness of the inner peripheral surfaces of the base tube 1 and the mating member 2.

According to some optional embodiments, the heat-shrink film 5 is a tubular structural component made of FEP (fluorinated ethylene propylene) material. The heat-shrink film 5 made of this material shrinks readily, has a smooth and non-adhesive surface, and enables easy peeling of the heat-shrink film 5 from the base tube 1, the mating member 2, and the interlocking sleeve 4 after thermal contraction is completed.

According to some optional embodiments, the shrink ratio of the heat-shrink film 5 is greater than or equal to 1.6:1, such that the heat-shrink film 5 can provide an appropriate contraction force during thermal contraction to promote infiltration of the material of the interlocking sleeve 4 into the base tube 1 and the mating member 2. At the same time, the shrink ratio of the heat-shrink film 5 should not be excessively large, so as to avoid applying excessive contraction pressure to the base tube 1 and the mating member 2, which would cause compressive deformation of the base tube 1 and the mating member 2.

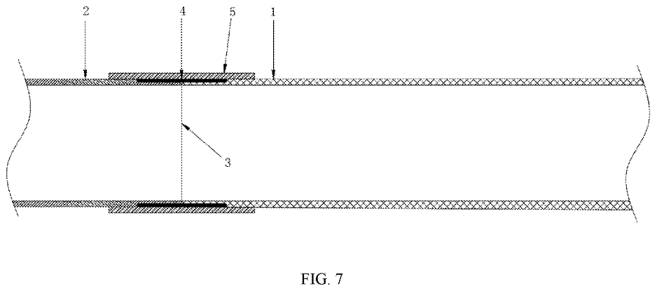

According to some optional embodiments, in the third step, as illustrated in FIG. 7, heating of the interlocking sleeve 4 and the heat-shrink film 5 is stopped when the inner peripheral surface of the heat-shrink film 5 at the portion covering the interlocking sleeve 4 comes into contact with the outer peripheral surfaces of the base tube 1 and the mating member 2, i.e., when the interlocking sleeve 4 has been fully embedded within the base tube 1 and the mating member 2 such that the outer peripheral surface of the interlocking sleeve 4 is flush with the outer peripheral surfaces of the base tube 1 and the mating member 2. At this point, the outer peripheral surface of the heat-shrink film 5 is a substantially smooth cylindrical surface. Stopping the heating of the interlocking sleeve 4 and the heat-shrink film 5 when the inner peripheral surface of the heat-shrink film 5 at the portion covering the interlocking sleeve 4 comes into contact with the outer peripheral surfaces of the base tube 1 and the mating member 2 effectively ensures that the interlocking sleeve 4 is exactly fully embedded within the base tube 1 and the mating member 2, and avoids continued heating that would cause the interlocking sleeve 4 to infiltrate excessively into the base tube 1 and the mating member 2, thereby resulting in a lack of sufficient material near the outer peripheral surfaces of the base tube 1 and the mating member 2 to maintain the connection, which would result in reduced connection strength of the product.

According to some optional embodiments, in the second step, a mandrel 6 is inserted through the base tube 1 and the mating member 2 to support the inner peripheral surfaces of the base tube 1 and the mating member 2. The length of the mandrel 6 is greater than the length of the interlocking sleeve 4, such that the mandrel 6 spans at least across the interlocking sleeve 4 within the base tube 1 and the mating member 2, so as to support the inner peripheral surfaces of the base tube 1 and the mating member 2 in the region surrounded by the interlocking sleeve 4 and to prevent compressive deformation of the base tube 1 and the mating member 2. In combination with a heat-shrink film 5 having a suitable shrink ratio, such as a shrink ratio of 1.6:1, the base tube 1 and the mating member 2 can be well protected and the inner peripheral surfaces of the base tube 1 and the mating member 2 will not undergo deformation.

Alternatively, in the second step, two mandrels 6 may be separately inserted into the base tube 1 and the mating member 2, respectively, to separately support the inner peripheral surfaces of the base tube 1 and the mating member 2. The manner of separately inserting two mandrels 6 is particularly suitable for configurations in which the base tube 1 and the mating member 2 are abutted together at an included angle.

According to some optional embodiments, in the third step, where the heat-shrink film 5 is not fitted over the outer periphery of the interlocking sleeve 4, taking an interlocking sleeve 4 made of Pebax material as an example, the interlocking sleeve 4 is generally heated directly by a heating device to 130° C., or alternatively, the ambient temperature surrounding the interlocking sleeve 4 is heated to indirectly heat the interlocking sleeve 4 to 130° C., thereby causing the interlocking sleeve 4 to undergo thermal contraction in the molten state.

According to some optional embodiments, where the heat-shrink film 5 is fitted over the outer periphery of the interlocking sleeve 4, taking a heat-shrink film 5 made of FEP material as an example, the heat-shrink film 5 is generally heated directly by a heating device to approximately 260° C., or alternatively, the ambient temperature surrounding the heat-shrink film 5 is heated to a range of 260° C. to 280° C. to indirectly heat the heat-shrink film 5 to approximately 260° C. The interlocking sleeve 4 is heated to the molten state through the heat-shrink film 5, thereby causing both the heat-shrink film 5 and the interlocking sleeve 4 to undergo thermal contraction in the molten state.

According to some optional embodiments, the manner of heating the interlocking sleeve 4 or the heat-shrink film 5 may be a direct heat blowing manner in which a hot air blower directly blows on the interlocking sleeve 4 or the heat-shrink film 5; or a traversing heating manner in which the base tube 1 and the mating member 2 are vertically fixed and an annular heating tube is fitted over the base tube 1 and the mating member 2 and moved vertically downward at a uniform speed to heat the interlocking sleeve 4 or the heat-shrink film 5; or a constant-temperature constant-time heating manner in which the base tube 1 and the mating member 2 are placed in an oven to heat the interlocking sleeve 4 or the heat-shrink film 5; or a conveyor manner in which the base tube 1 and the mating member 2 are laid flat on a conveyor belt and conveyed at a uniform speed through a furnace to heat the interlocking sleeve 4 or the heat-shrink film 5.

In another aspect, the present application provides a tube joint structure, which specifically adopts the following configuration:

The tube joint structure of the present embodiment is produced by the polymeric material fusion joining method according to any of the above embodiments.

As illustrated in FIG. 4, the tube joint structure includes a base tube 1, a mating member 2, and an interlocking sleeve 4. The mating member 2 is a tube serving to extend the base tube 1.

The base tube 1 and the mating member 2 are abutted against each other, with a joint seam 3 formed at the abutment junction of the base tube 1 and the mating member 2. At least a portion of the material of the interlocking sleeve 4, in the molten state, is embedded from the outer side into the entire circumferential wall of the base tube 1 and the mating member 2 to form an interlocking structure by means of thermal contraction or further under the pressure of the heat-shrink film 5, and fills the joint seam 3 from the outer side to seal the joint seam 3.

According to some optional embodiments, the interlocking sleeve 4, as illustrated in FIGS. 4 and 7, is fully embedded within the base tube 1, the mating member 2, and the joint seam 3 formed at the abutment junction of the base tube 1 and the mating member 2. The wall thickness of the interlocking sleeve 4 is less than the wall thickness of the base tube 1 and the mating member 2, such that the outer peripheral surface of the interlocking sleeve 4 is flush with the outer peripheral surfaces of the base tube 1 and the mating member 2, while at the same time the inner peripheral surface of the interlocking sleeve 4 is located radially outward of the inner peripheral surfaces of the base tube 1 and the mating member 2.

According to some optional embodiments, the interlocking sleeve 4 is partially embedded within the base tube 1, the mating member 2, and the joint seam 3 formed at the abutment junction of the base tube 1 and the mating member 2, and the inner peripheral surface of the interlocking sleeve 4 is located radially outward of the inner peripheral surfaces of the base tube 1 and the mating member 2.

In yet another aspect, the present application provides an endoscope, which specifically adopts the following configuration:

The endoscope of the present embodiment includes an endoscope control section and an insertion section connected to the control section. The insertion section has a plurality of the above tube joint structures.

According to some optional embodiments, the tube joint structure is formed in an outer sheath of the insertion section, such that the outer sheath is extended by means of the tube joint structure.

According to some optional embodiments, the tube joint structure is formed in an instrument channel tube, such that the instrument channel tube is extended by means of the tube joint structure.

According to some optional embodiments, the tube joint structure is formed between a distal end fitting and an articulating spine, the distal end fitting and the articulating spine being specifically made of polymeric materials, such that the distal end fitting and the articulating spine of the actively deflectable section of the insertion section of the endoscope are connected by means of the tube joint structure.

The foregoing descriptions are merely specific embodiments of the present disclosure, but the scope of protection of the present disclosure is not limited thereto. Any variations or substitutions readily conceivable by those skilled in the art within the technical scope disclosed by the present disclosure shall fall within the scope of protection of the present disclosure.

What is claimed is:

1. A polymeric material fusion joining method, comprising:
a first step of abutting a base tube and a mating member that are to be joined to each other so as to form a joint seam at an abutment junction thereof;
a second step of fitting a tubular interlocking sleeve, which spans across the joint seam, over an outer periphery of the base tube and the mating member;
wherein the interlocking sleeve is made of a polymeric material; and the method further comprises:
a third step of heating the interlocking sleeve to a molten state to cause thermal contraction thereof, such that at least a portion of the material of the interlocking sleeve infiltrates into an entire circumferential wall of the base tube and the mating member that are not in a fully molten state, and fills the joint seam over an entire circumference thereof, so as to maintain smoothness of inner peripheral surfaces of the base tube and the mating member; and
a fourth step of allowing the interlocking sleeve to cool and solidify, using only the interlocking sleeve as a connecting member so as to form an interlocking engagement with the base tube and the mating member, respectively, and to seal the joint seam.

2. The polymeric material fusion joining method according to claim 1, wherein the interlocking sleeve is a structural component made of Pebax material; and/or
the base tube is a structural component made of any one of Pebax, PTFE, LDPE, and TPU; and/or
the mating member is a structural component made of any one of Pebax, PTFE, LDPE, and TPU.

3. The polymeric material fusion joining method according to claim 1, wherein in the third step, the interlocking sleeve is heated by heating an ambient environment surrounding the interlocking sleeve.

4. The polymeric material fusion joining method according to claim 1, wherein in the third step, all of the material of the interlocking sleeve infiltrates into the entire circumferential wall of the base tube and the mating member and fills the joint seam over the entire circumference thereof, such that an outer peripheral surface of the interlocking sleeve is flush with outer peripheral surfaces of the base tube and the mating member.

5. The polymeric material fusion joining method according to claim 1, wherein:
in the second step, a tubular heat-shrink film spanning across the joint seam is fitted over an outer periphery of the interlocking sleeve, wherein a melting point of the heat-shrink film is higher than a melting point of the interlocking sleeve;
in the third step, the heat-shrink film is heated to cause thermal contraction thereof, so as to apply a contraction force to the interlocking sleeve through the contraction;
in the fourth step, the heat-shrink film is allowed to cool and solidify; and
the polymeric material fusion joining method further comprises a fifth step of peeling off the heat-shrink film.

6. The polymeric material fusion joining method according to claim 5, wherein in the second step, a heat-shrink film having a length greater than a length of the interlocking sleeve is selected such that the heat-shrink film completely covers the interlocking sleeve in an axial direction; and/or
the heat-shrink film is a structural component made of FEP material.

7. The polymeric material fusion joining method according to claim 5, wherein a shrink ratio of the heat-shrink film is greater than or equal to 1.6:1; and/or
in the third step, heating of the interlocking sleeve and the heat-shrink film is stopped when an inner peripheral surface of the heat-shrink film at a portion thereof covering the interlocking sleeve comes into contact with outer peripheral surfaces of the base tube and the mating member.

8. The polymeric material fusion joining method according to claim 1, wherein in the second step, a mandrel spanning at least across the interlocking sleeve is inserted through the base tube and the mating member to support inner peripheral surfaces of the base tube and the mating member; and/or
the mating member is a tube, a tube fitting, or an end cap.

9. A tube joint structure, produced by the method according to claim 1, comprising an interlocking sleeve, and a base tube and a mating member abutted against each other, wherein the base tube and the mating member form a joint seam at an abutment junction thereof;
at least a portion of the material of the interlocking sleeve is embedded from an outer side into the entire circumferential wall of the base tube and the mating member to form an interlocking structure, and fills the joint seam from the outer side to seal the joint seam.

10. An endoscope, comprising an insertion section, wherein the insertion section has a plurality of tube joint structures according to claim 9.
